(19) United States
(12) Patent Application Publication
(10) Pub. No.: US 2016/0215244 A1
(43) Pub. Date: Jul. 28, 2016

(54) Method for Production of Brewers Wort

(71) Applicant: NOVOZYMES A/S, Bagsvaerd (DK)

(72) Inventors: Alexander Mauch, Copenhagen (DK); Niels Elvig, Holte (DK); Jens Ekloef, Copenhagen (DK); Kristian Bertel Roemer M. Krogh, Bagsvaerd (DK)

(73) Assignee: Novozymes A/S, Bagsvaerd (DK)

(21) Appl. No.: 14/915,772

(22) PCT Filed: Sep. 4, 2014
(86) PCT No.: PCT/EP2014/068820

§ 371 Date: Mar. 1, 2016

(30) Foreign Application Priority Data
Sep. 5, 2013 (EP) 13183102.6

(51) Int. Cl. C12C 5/00; C12C 7/165; C12N 9/24; C12C 7/047

(52) U.S. Cl. CPC C12C 5/004; C12C 7/047; C12C 7/165; C12N 9/2402; C12Y 302/01055

(57) ABSTRACT

A method of reducing the viscosity in a brewing process comprising the steps of: (a) preparing a mash from malt and adjunct, and (b) adding an arabinofuranosidase GH43 to the mash.

### REFERENCE TO A SEQUENCE LISTING

This application contains a Sequence Listing in computer readable form. The computer readable form is incorporated herein by reference.

### FIELD OF INVENTION

The present invention relates to a method of reducing the viscosity in a brewing process comprising malt and adjunct.

### BACKGROUND OF THE INVENTION

Brewing processes are well known in the art. It normally involves the steps of malting, mashing, mash filtration, wort boiling, and fermentation/maturation.

Briefly, during malting, grains are allowed to germinate and then dried and optionally roasted. The malting process causes the activation of a number of enzymes in the grain which can convert the starch in the grain to sugar.

Prior to mashing, the malt is crushed to form a grist, which is mixed with water to form a mash. The grist may also contain one or more adjuncts. Mashing is the process of converting starch in the mash into fermentable and un-fermentable sugars. The mashing process is normally conducted over a period of time at various temperatures in order to activate the endogenous enzymes responsible for the degradation of proteins and carbohydrates.

Exogenous enzymes may be added during the mashing process to speed up the reactions and enable better control over the brewing process. Towards the end of mashing, the temperature may be raised to 75-80° C. After the mashing, the resulting liquid is strained from the grains in a filtration step such as mash filtration or lautering. The liquid resulting from the filtration step is known as the wort. The wort, which is rich in sugars, is then boiled with hops, cooled, and then fermented to ethanol using yeast. The resulting beer is conditioned for a period of a week up to several months and then packaged.

Important non-starch polysaccharides present in cereal grains are beta-glucan and arabinoxylan. The cereal endosperm cell wall of various cereals comprises typically 20-75% beta-glucan, 20-75% arabinoxylan, and 5% remaining protein with a small amount of cellulose, glucomannan and phenolic acids. Long chains of arabinoxylans, and to a lesser degree beta-glucan, which have not been modified due to enzymatic hydrolysis, may cause formation of gels when solubilised in water, and these gels will strongly increase wort viscosity and reduce filterability.

WO 97/42301 describes the use of alpha-L-arabinofuranosidases (GH51 and GH54) in a process for preparing a wort.

Of late, there is a dramatic changing in raw material prices caused by increased demand for grains, global water shortage, changing weather patterns, etc. This has forced the brewing industry to focus on production efficiency as well as raw material savings.

There exists a need for improved filtration processes in brewing which will bring down costs and/or increase production efficiency.

### DRAWINGS

FIG. 1 shows the comparison of arabinofuranosidases belonging to family GH51 and GH43 regarding viscosity reduction in high gravity mashing with barley and malt. Left side: Dosage response curves. Right side: Viscosity reduction expressed in percent of control.

FIG. 2 shows the comparison of arabinofuranosidases belonging to family GH51 and GH43 regarding viscosity reduction in high gravity mashing with wheat and malt. Left side: Dosage response curves. Right side: Viscosity reduction expressed in percent of control.

### SUMMARY OF THE INVENTION

The inventors have found that the viscosity of a brewing process may be significantly reduced by adding an arabinofuranosidase GH43 to the mash, so we claim:

A method of reducing the viscosity in a brewing process comprising the steps of:
(a) preparing a mash from malt and adjunct, and
(b) adding an arabinofuranosidase GH43 to the mash.

In one aspect, the invention relates to a method, wherein the adjunct is selected from the group consisting of barley and wheat.

In one aspect, the invention relates to a method, wherein additionally a beta-glucanase is added to the mash.

In one aspect, the invention relates to a method, wherein additionally a xylanase is added to the mash.

In one aspect, the invention relates to a method, wherein the arabinofurosidase GH43 has at least 70% identity to the sequence shown in SEQ ID NO: 1.

In one aspect, the invention relates to a method, wherein the mash has a water/grist ratio of from 2.0:1.0 to 3.0:1.0 (w/w).

In one aspect, the invention relates to a method, wherein the adjunct is at least 10% (w/w) of the total weight of adjunct and malt.

In one aspect, the invention relates to a method, wherein the mashing comprises an incubation step at a temperature in the range of from 45° C. to 60° C.

In one aspect, the invention relates to a method, wherein additionally a protease is added to the mash.

In one aspect, the invention relates to a method, wherein additionally a pullulanase is added to the mash.

In one aspect, the invention relates to a method, wherein additionally a lipase is added to the mash.

In one aspect, the invention relates to a method, wherein additionally an alpha-amylase is added to the mash.

In one aspect, the invention relates to a method, wherein the mash is filtered to obtain a wort.

In one aspect, the invention relates to a method, wherein the wort is fermented to obtain a beer.

In one aspect, the invention relates to a method, wherein the arabinofuranosidase GH43 is added in an amount of from 0.5 to 10.0 mg enzyme protein per kg grist.

In one aspect, the invention relates to a method, wherein the arabinofuranosidase GH43 has activity towards di-substituted xyloses.

### DETAILED DESCRIPTION OF THE INVENTION

### Definitions

Throughout this disclosure, various terms that are generally understood by those of ordinary skill in the arts are used. Several terms are used with specific meaning, however, and are meant as defined by the following.

As used herein the term “grist” is understood as the starch or sugar containing material that is the basis for beer production, e.g., the barley malt and the adjunct. Generally, the grist does not contain any added water.

The term “malt” is understood as any malted cereal grain, in particular barley.

The term “adjunct” is understood as the part of the grist which is not barley malt. The adjunct may be any starch rich plant material, e.g., unmalted grain, such as, but not limited to, barley, corn, rice, sorghum, and wheat. Adjunct also includes readily fermentable sugar and/or syrup.

The starch of some of the adjuncts has a relatively low gelatinization temperature which enables them to be mashed in together with the malt, whereas other adjuncts such as rice, corn and sorghum have a higher gelatinization temperature, such adjuncts are typically separately cooked and liquefied with an alpha-amylase before they are added to the mash.

The term “mash” is understood as a starch containing slurry comprising crushed barley malt, crushed unmalted grain, other starch containing material, or a combination hereof, steeped in water to make wort.

The term “wort” is understood as the unfermented liquor run-off following extracting the grist during mashing.

The term “spent grains” is understood as the drained solids remaining when the grist has been extracted and the wort separated.

The term “beer” is here understood as fermented wort, i.e., an alcoholic beverage brewed from malt, adjunct and hops. The term “beer” as used herein is intended to cover at least beer prepared from mashes prepared from a mixture of malted and unmalted cereals. The term “beer” also covers beers prepared with adjuncts, and beers with all possible alcohol contents.

### Sequence Identity:

The relatedness between two amino acid sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

### Wort Production According to the Invention

The present invention relates to a method of reducing the viscosity in a brewing process comprising the steps of:
(a) preparing a mash from malt and adjunct, and
(b) adding an arabinofuranosidase GH43 to the mash.

According to the invention, the arabinofuranosidase GH43 is added to the mash, whereby the viscosity is reduced, and any filtration step downstream in the brewing process will be faster.

The malt is preferably derived from one or more of the grains selected from the list consisting of corn, barley, wheat, rye, sorghum, millet and rice. Preferably, the malt is barley malt.

In addition to the malt, the grist according to the invention comprises one or more adjuncts such as unmalted corn, or other unmalted grain, such as barley, wheat, rye, oat, corn, rice, milo, millet and/or sorghum, or raw and/or refined starch and/or sugar containing material derived from plants like wheat, rye, oat, corn, rice, milo, millet, sorghum, potato, sweet potato, cassava, tapioca, sago, banana, sugar beet and/or sugar cane. According to the invention, adjuncts may be obtained from tubers, roots, stems, leaves, legumes, cereals and/or whole grain.

According to the invention, an adjunct selected from the group consisting of barley and wheat is preferred.

Prior to forming the mash, the malt and/or adjunct is preferably milled and most preferably dry or wet milled.

According to the invention, the adjunct is at least 10% (w/w) of the total weight of the adjunct and the malt, e.g., the adjunct is at least 15% (w/w) of the total weight of the adjunct and the malt, e.g., the adjunct is at least 20% (w/w) of the total weight of the adjunct and the malt, e.g., the adjunct is at least 25% (w/w) of the total weight of the adjunct and the malt, e.g., the adjunct is at least 30% (w/w) of the total weight of the adjunct and the malt, e.g., the adjunct is at least 35% (w/w) of the total weight of the adjunct and the malt, e.g., the adjunct is at least 40% (w/w) of the total weight of the adjunct and the malt, e.g., the adjunct is at least 45% (w/w) of the total weight of the adjunct and the malt, e.g., the adjunct is at least 50% (w/w) of the total weight of the adjunct and the malt, e.g., the adjunct is at least 55% (w/w) of the total weight of the adjunct and the malt, e.g., the adjunct is at least 60% (w/w) of the total weight of the adjunct and the malt, e.g., the adjunct is at least 65% (w/w) of the total weight of the adjunct and the malt, e.g., the adjunct is at least 70% (w/w) of the total weight of the adjunct and the malt, e.g., the adjunct is at least 75% (w/w) of the total weight of the adjunct and the malt, e.g., the adjunct is at least 80% (w/w) of the total weight of the adjunct and the malt, e.g., the adjunct is at least 85% (w/w) of the total weight of the adjunct and the malt, e.g., the adjunct is at least 90% (w/w) of the total weight of the adjunct and the malt, e.g., the adjunct is at least 95% (w/w) of the total weight of the adjunct and the malt.

In accordance with one aspect of the invention, the mash is obtained by grounding a grist comprising malt and adjunct. The water added to the grist may be preheated in order for the mash to attain the desired mash temperature at the moment of mash forming.

The present invention is especially useful for a low water/grist ratio, so according to the invention, the mash has a water/grist ratio of from 2.0:1.0 to 3.0:1.0 (w/w), e.g., the mash has a water/grist ratio of from 2.1:1.0 to 3.0:1.0 (w/w), e.g., the mash has a water/grist ratio of from 2.2:1.0 to 3.0:1.0 (w/w), e.g., the mash has a water/grist ratio of from 2.3:1.0 to 3.0:1.0 (w/w), e.g., the mash has a water/grist ratio of from 2.4:1.0 to 3.0:1.0 (w/w), e.g., the mash has a water/grist ratio of from 2.5:1.0 to 3.0:1.0 (w/w).

The temperature profile of the mashing process may be a profile from a conventional mashing process wherein the temperatures are set to achieve optimal degradation of the grist dry matter by the malt enzymes.

The mashing process generally applies a controlled stepwise increase in temperature, where each step favors one enzymatic action over the other. Mashing temperature profiles are generally known in the art. In one aspect of the present invention, the mashing comprises at least one incubation step at a temperature in the range of from 45° C. to 60° C., wherein the arabinofuranosidase GH43 is active.

An example of a mashing temperature profile useful according to the present invention is 52° C. (20 min); 64° C. (40 min); 72° C. (20 min); 78° C. (5 min); wherein a heating rate of 1° C./min. has been used.

In one aspect, the pH of the mash is in the range of about 4.6 to about 6.4. In another aspect, the pH is in the range of about 4.6 to 6.2, such as in the range between pH about 4.8 to about 6.0, preferably in the range between pH about 5.0 to about 6.0, more preferably in the range between pH about 5.0 to about 5.6, even more preferably in the range between pH about 5.0 to about 5.4.

Obtaining the wort from the mash typically includes straining the wort from the spent grains. Hot water may be run through the spent grains to rinse out, or sparge, any remaining extract from the grist.

Following the separation of the wort from the spent grains of the grist, the wort may be used as it is, or it may be dewatered to provide a concentrated and/or dried wort. The concentrated and/or dried wort may be used as brewing extract, as malt extract flavoring, for non-alcoholic malt beverages, malt vinegar, breakfast cereals, for confectionary etc.

In a preferred embodiment, the wort is fermented to produce an alcoholic beverage, preferably a beer, e.g., ale, strong ale, bitter, stout, porter, lager, export beer, malt liquor, barley wine, happoushu, high-alcohol beer, low-alcohol beer, low-calorie beer or light beer. Fermentation of the wort may include pitching the wort with a yeast slurry comprising fresh yeast, i.e., yeast not previously used for the invention or the yeast may be recycled yeast. The yeast applied may be any yeast suitable for beer brewing, especially yeasts selected from Saccharomyces spp. such as S. cerevisiae and S. uvarum, including natural or artificially produced variants of these organisms. The methods for fermentation of wort for production of beer are well known to the person skilled in the art.

### Enzymes

In one aspect, the arabinofuranosidase GH43 is introduced at the beginning of mashing. In another aspect, the arabinofuranosidase GH43 is introduced during mashing.

In one aspect, the invention comprises adding both an arabinofuranosidase GH43 and a beta-glucanase to the mash; in particular an arabinofuranosidase GH43 and a beta-glucanase GH5 to the mash; preferably an arabinofuranosidase GH43 and a beta-glucanase GH5_5 to the mash.

In one aspect, the invention comprises adding both an arabinofuranosidase GH43 and a xylanase to the mash; in particular an arabinofuranosidase GH43 and a xylanase GH10 to the mash.

In one aspect, the invention comprises adding an arabinofuranosidase GH43, a beta-glucanase, and a xylanase to the mash.

In one aspect, the invention comprises adding an arabinofuranosidase GH43, a beta-glucanase GH5 and a xylanase GH10, in particular an arabinofuranosidase GH43, a beta-glucanase GH5_5 and a xylanase GH10.

In another preferred embodiment, a further enzyme(s) is added to the mash, said enzyme(s) including but not limited to a protease, a pullulanase, a lipase, and an alpha-amylase.

The enzymes to be applied in the present invention should be selected for their ability to retain sufficient activity at the process temperature of the processes of the invention, as well as for their ability to retain sufficient activity under moderate acid pH in the mash and should be added in effective amounts. The enzymes may be derived from any source, preferably from a plant or algae, and more preferably from a microorganism, such as from bacteria or fungi.

### Arabinofuranosidase GH43

The numbering of Glycoside Hydrolase Families applied in this disclosure follows the concept of Coutinho, P. M. & Henrissat, B. (1999) CAZy—Carbohydrate Active Enzymes server at URL: http://afmb.cnrs-mrs.fr/˜cazy/CAZY/index.html, or alternatively Coutinho, P. M. & Henrissat, B. 1999; The modular structure of cellulases and other carbohydrate-active enzymes: an integrated database approach, in “Genetics, Biochemistry and Ecology of Cellulose Degradation”., K. Ohmiya, K. Hayashi, K. Sakka, Y. Kobayashi, S. Karita and T. Kimura eds., Uni Publishers Co., Tokyo, pp. 15-23, and Bourne, Y. & Henrissat, B. 2001; Glycoside hydrolases and glycosyltransferases: families and functional modules, Current Opinion in Structural Biology 11:593-600.

In a preferred embodiment, arabinofuranosidase GH43, or also called alpha-L-arabinofuranosidase of GH43, has activity towards di-substituted xyloses.

The arabinofuranosidase GH43 may be of microbial origin, e.g., derivable from a strain of a filamentous fungus (e.g., Humicola, Aspergillus, Trichoderma, Fusarium, Penicillum) or from a bacteria (e.g., Bacillus, Bifidobacterium).

Preferably, the arabinofuranosidase GH43 is derived from Humicola insolens. Most preferably the arabinofuranosidase GH43 has at least 70% identity to the sequence shown in SEQ ID NO:1; preferably the arabinofuranosidase GH43 has at least 75% identity to the sequence shown in SEQ ID NO:1; preferably the arabinofuranosidase GH43 has at least 80% identity to the sequence shown in SEQ ID NO:1; preferably the arabinofuranosidase GH43 has at least 85% identity to the sequence shown in SEQ ID NO:1; preferably the arabinofuranosidase GH43 has at least 90% identity to the sequence shown in SEQ ID NO:1; preferably the arabinofuranosidase GH43 has at least 91% identity to the sequence shown in SEQ ID NO:1; preferably the arabinofuranosidase GH43 has at least 92% identity to the sequence shown in SEQ ID NO:1; preferably the arabinofuranosidase GH43 has at least 93% identity to the sequence shown in SEQ ID NO:1; preferably the arabinofuranosidase GH43 has at least 94% identity to the sequence shown in SEQ ID NO:1; preferably the arabinofuranosidase GH43 has at least 95% identity to the sequence shown in SEQ ID NO:1; preferably the arabinofuranosidase GH43 has at least 96% identity to the sequence shown in SEQ ID NO:1; preferably the arabinofuranosidase GH43 has at least 97% identity to the sequence shown in SEQ ID NO:1; preferably the arabinofuranosidase GH43 has at least 98% identity to the sequence shown in SEQ ID NO:1; preferably the arabinofuranosidase GH43 has at least 99% identity to the sequence shown in SEQ ID NO:1; preferably the arabinofuranosidase GH43 has 100% identity to the sequence shown in SEQ ID NO:1.

The arabinofuranosidase GH43 may also be derived from Bifidobacterium adolescenti. More preferably, the arabinofuranosidase GH43 is the enzyme described by Van Laere, 1997, in Appl. Microbiol. Biotechnol, 47, 231-235 and/or by Van den Broek, 2005, in Applied Microbiology and Biotechnology.

The arabinofuranosidase GH43 may be added in an amount of 0.5-20 mg enzyme protein per kg grist; preferably 0.5-15 mg enzyme protein per kg grist; and even more preferably 0.5-10 mg enzyme protein per kg grist.

### Arabinofuranosidase GH51

An arabinofuranosidase GH51, or also called alpha-L-arabinofuranosidase of GH51, has activity towards single-substituted xyloses. It may be of microbial origin, e.g., derivable from a strain of a filamentous fungus (e.g., Meripilus, Humicola, Aspergillus, Trichoderma, Fusarium, Penicillum) or from a bacteria (e.g. Bacillus).

Preferably, the enzyme is an arabinofuranosidase of GH51 derived from Meripilus giganteus. Most preferably, the arabinofuranosidase of GH51 is the polypeptide shown as SEQ ID NO:2.

### Beta-Glucanase

In a further embodiment, a beta-glucanase (EC 3.2.1.4.) is added to the mash. Beta-glucanase is also termed cellulase and may be of fungal origin such as from Aspergillus, e.g., Aspergillus orzyae or Aspergillus niger or from a bacteria such as Bacillus, e.g., Bacillus subtilis.

In particular, the beta-glucanase may be derived from Thermoascus, in particular from Thermoascus aurantiacus. Most preferably, the beta-glucanase is a GH5 glucanase; in particular a GH5_5 glucanase.

### Xylanase

In a further embodiment, a xylanase is added to the mash. In one aspect, xylanase activity is provided by a xylanase from glycosyl hydrolase family 10. In one aspect, the xylanase has at least 50%, more preferably at least 60%, more preferably at least 70%, more preferably at least 80%, more preferably at least 85%, more preferably at least 90%, more preferably at least 91%, more preferably at least 92%, more preferably at least 93%, more preferably at least 94% more preferably at least 95%, more preferably at least 96%, more preferably at least 97% more preferably at least 98%, and most preferably at least 99% or even 100% identity to the xylanase described in WO 94/21785. In another aspect, the xylanase is Shearzyme™ from Novozymes NS.

In a preferred embodiment, the xylanase is derived from Aspergillus aculeatus, especially a family GH10 xylanase from Aspergillus aculeatus.

### Protease

In a further embodiment, a protease is added to the mash. Suitable proteases include microbial proteases, such as fungal and bacterial proteases. Preferred proteases are acidic proteases, i.e., proteases characterized by the ability to hydrolyze proteins under acidic conditions below pH 7. The proteases are responsible for reducing the overall length of high-molecular-weight proteins to low-molecular-weight proteins in the mash. The low-molecular-weight proteins are a necessity for yeast nutrition and the high-molecular-weight-proteins ensure foam stability. Thus it is well-known to the skilled person that protease should be added in a balanced amount which at the same time allows ample free amino acids for the yeast and leaves enough high-molecular-weight-proteins to stabilize the foam. In one aspect, the protease activity is provided by a proteolytic enzymes system having a suitable FAN generation activity including endo-proteases, exopeptidases or any combination hereof, preferably a metallo-protease. Preferably, the protease has at least 50%, more preferably at least 60%, more preferably at least 70%, more preferably at least 80%, more preferably at least 85%, more preferably at least 90%, more preferably at least 91%, more preferably at least 92%, more preferably at least 93%, more preferably at least 94%, more preferably at least 95% more preferably at least 96%, more preferably at least 97% more preferably at least 98%, and most preferably at least 99% or even 100% identity to the amino acid sequence shown in SEQ ID NO:6 described in WO 99/67370. In another aspect, the protease is Neutrase™ available from Novozymes NS.

### Pullulanase

In a further embodiment, a pullulanase (EC 3.2.1.41) is added to the mash. Pullulanases catalyze the hydrolysis of (1->6)-alpha-D-glucosidic linkages in pullulan, amylopectin and glycogen, and in the alpha- and beta-limit dextrins of amylopectin and glycogen.

The pullulanase according to the present invention is preferably a pullulanase from e.g. Pyrococcus or Bacillus, such as Bacillus acidopullulyticus e.g. the one described in Kelly et al., 1994, FEMS Microbiol. Letters 115: 97-106, or a pullulanase available from Novozymes NS as Promozyme 400L. The pullulanase may also be from Bacillus naganoencis, or Bacillus deramificans e.g. such as derived from Bacillus deramificans (U.S. Pat. No. 5,736,375).

Most preferably the pullulanase is derived from Bacillus acidopullulyticus. A preferred pullulanase is a pullulanase having an amino acid sequence which is at least 50%, such as at least 55%, such as at least 60%, such as at least 65%, such as at least 66%, such as at least 70%, such as at least 75%, such as at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or even 100% identical to the sequence of the pullulanase 3 disclosed in WO 2009/075682.

### Lipase

In a further embodiment, a lipase (EC 3.2.1.41) is added to the mash. In one embodiment, the lipase activity is provided by a lipase having activity to triglycerides and/or galactolipids and/or phospholipids. Preferably, the lipase activity is provided by a lipase from Fusarium (including F. oxysporum and F. heterosporum), Aspergillus (including A. tubigensis), Rhizopus (including R. oryzae) or Thermomyces (including T. lanuginosus) or a variant of these. An example is Lipopan X (Lipopan Xtra), a variant of the Thermomyces lanuginosus lipase with the substitutions G91A+D96W+E99K+P256V+G263Q+L264A+1265T+G266D+T267A+L269N+270A+271G+272G+273F (+274S), described in WO2004099400A2. In another aspect, the lipase is a lipase/phospholipase from Fusarium oxysporum, described in EP 869167, available from Novozymes NS as Lipopan™ F. In a preferred embodiment of the invention, the lipase is Lipozyme TL® or Lipolase®; this lipase has a significantly good effect on filtration speed and haze reduction and is available from Novozymes NS, Denmark. The lipase may also be Lipex®, a variant of Lipozyme, available from Novozymes NS Denmark. The lipases degrade the lipid from barley, e.g., the triglycerides into partial glycerides and free fatty acids. This leads to a lower turbidity and much improved mash filtration and lautering properties.

### Alpha-Amylase

In a further embodiment, an alpha-amylase (EC 3.2.1.1) is added to the mash. A particular alpha-amylase enzyme to be used in the processes and/or compositions of the invention may be a Bacillus alpha-amylase. Well-known Bacillus alpha-amylases include alpha-amylase derived from a strain of B. licheniformis, B. amyloliquefaciens, and B. stearothermophilus. In one aspect of the present invention, a useful Bacillus alpha-amylase is an alpha-amylase as defined in WO 99/19467 on page 3, line 18 to page 6, line 27. Preferably the alpha-amylase has at least 50%, more preferably at least 60%, more preferably at least 70%, more preferably at least 80%, preferably at least 85%, more preferably at least 90%, preferably at least 91%, preferably at least 92%, preferably at least 93%, preferably at least 94%, more preferably at least 95%, preferably at least 96%, preferably at least 97%, more preferably at least 98%, and most preferably at least 99% identity to the amino acid sequence shown in the amino acid sequence disclosed as SEQ ID NO: 3 in WO 99/19467 with the mutations: 1181*+G182*+N193F. Also contemplated is the alpha-amylase Termamyl™ SC from Novozymes NS. Another alpha-amylase to be used in the processes of the invention may be any fungal alpha-amylase, e.g., an alpha-amylase derived from a species within Aspergillus, and preferably from a strain of Aspergillus niger.

The invention is further illustrated in the following examples, which are not intended to be in any way limiting to the scope of the invention as claimed.

### Example 1

### Comparison of Arabinofuranosidases (AraF) Belonging to Family GH43 Versus Family GH51 in their Capability of Reducing Wort Viscosity when Added into a Mash Comprising Barley Malt and Barley

Malt wort was made in the following way:

48.0 g grinded barley malt (0.2 mm) and 32.0 g barley were added to a mashing beaker together with 197 ml H₂O (54° C.) and 3.0 ml CaCl2 solution (11.0 g CaCl₂.2H₂O/500 ml H₂O).

Arabinofuranosidases belonging to family GH43 (SEQ ID NO:1) and GH51 (SEQ ID NO:2) were individually added at the beginning of mashing (52° C.) in dosages of 2.5, 5, and 10 mg EP/kg grist, respecitvely.

The following mashing profile was used: 52° C. (20 min), 64° C. (40 min), 72° C. (20 min), 78° C. (5 min), cooling to 20° C. The heating rate between mashing rests was 1° C./min.

After mashing, evaporation losses were corrected by addition of water aiming for final weight of 280 g. The mash was filtered through a paper filter (Whatman 597%) placed in a plastic funnel. Filtrate (wort) was analyzed for density (Beeranalyzer, Anton Paar, Graz, Austria) and dynamic viscosity (Microviscosimeter, Anton Paar, Graz, Austria) at 20° C. Dynamic viscosity η=f(density) was standardized to the level of 24.2° P using the hyperbolic function described in Mitteleuropaische Brautechnische Analysenkommission (MEBAK) “Raw Material” (2011), Chapter 3.1.4.4.

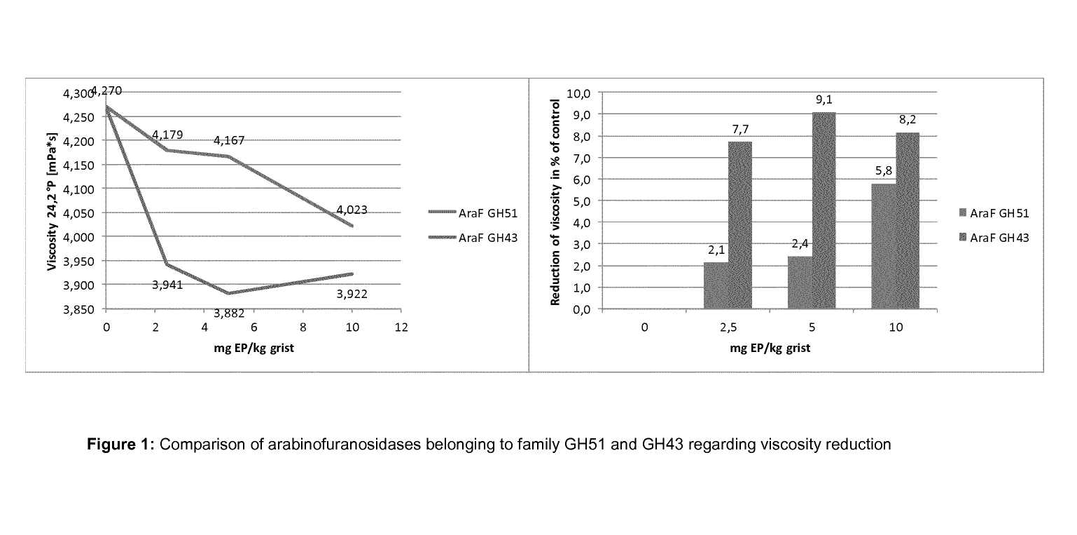

FIG. 1 shows the comparison of arabinofuranosidases belonging to family GH51 and GH43 regarding viscosity reduction in high gravity mashing with barley and malt. Left side: Dosage response curves; Right side: Viscosity reduction expressed in percent of control. The figure shows that at an enzyme level of 2.5 mg enzyme protein per kg grist, the viscosity reduction is 2.1% when AraF GH51 is used, whereas the the viscosity reduction is 7.7% when AraF GH43 is used. This shows that AraF GH43 is surprisingly good in high gravity mashing.

### Example 2

### Comparison of Arabinofuranosidases (AraF) Belonging to Family GH43 Versus Family GH51 in their Capability of Reducing Wort Viscosity when Added into a Mash Comprising Barley Malt and Wheat

Malt wort was made in the following way:

48.0 g grinded barley malt (0.2 mm) and 32.0 g wheat were added to a mashing beaker together with 197 ml H₂O (54° C.) and 3.0 ml CaCl2 solution (11.0 g CaCl₂.2H₂O/500 ml H₂O).

Arabinofuranosidases belonging to family GH43 (SEQ ID NO:1) and GH51 (SEQ ID NO:2) were individually added at the beginning of mashing (52° C.) in dosages of 2.5, 5, and 10 mg EP/kg grist, respecitvely.

The following mashing profile was used: 52° C. (20 min), 64° C. (40 min), 72° C. (20 min), 78° C. (5 min), cooling to 20° C. The heating rate between mashing rests was 1° C./min.

After mashing, evaporation losses were corrected by addition of water aiming for final weight of 280 g. The mash was filtered through a paper filter (Whatman 597%) placed in a plastic funnel. Filtrate (wort) was analyzed for density (Beeranalyzer, Anton Paar, Graz, Austria) and dynamic viscosity (Microviscosimeter, Anton Paar, Graz, Austria) at 20° C. Dynamic viscosity η=f(density) was standardized to the level of 24.2° P using the hyperbolic function described in Mitteleuropaische Brautechnische Analysenkommission (MEBAK) “Raw Material” (2011), Chapter 3.1.4.4.

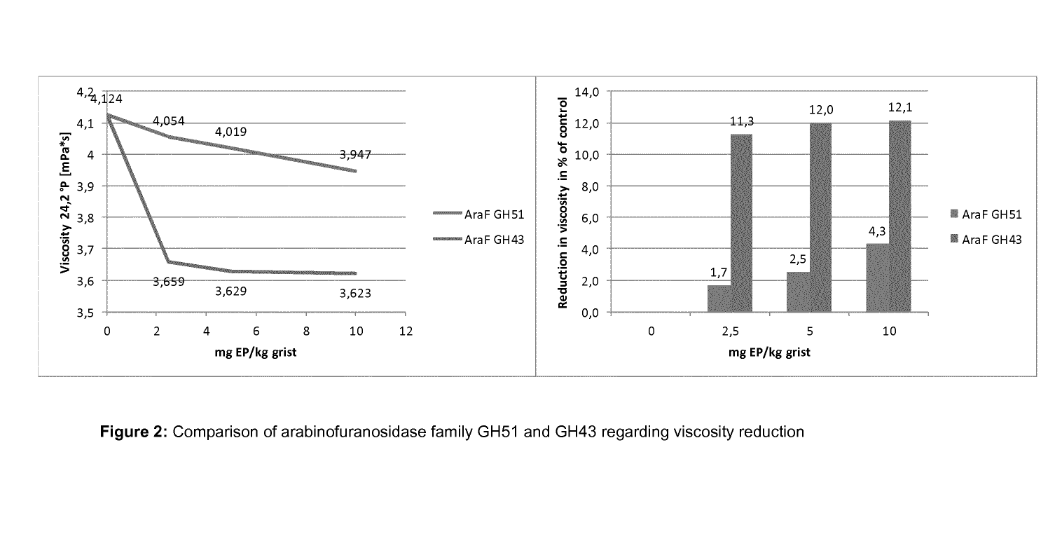

FIG. 2 shows the comparison of arabinofuranosidases belonging to family GH51 and GH43 regarding viscosity reduction in high gravity mashing with wheat and malt. Left side: Dosage response curves; Right side: Viscosity reduction expressed in percent of control. The figure shows that at an enzyme level of 2.5 mg enzyme protein per kg grist, the viscosity reduction is 1.7% when AraF GH51 is used, whereas the the viscosity reduction is 11.3% when AraF GH43 is used. This shows that AraF GH43 is surprisingly good in high gravity mashing.

### Example 3

### Impact of Arabinofuranosidase Fam. GH43 from H. insolens (SEQ ID:1) Application in Mashing on the Subsequent Mash Filtration

### Material and Methods

Un-malted barley (184 g) and Pilsner malt (276 g) were milled using a hammer-mill equipped with a 0.2 mm screen. Calcium-chloride was added to de-salted water to achieve a final concentration of 60 ppm Ca²⁺-ions. After heating the water to 51° C., the grist (room temperature) was added. When reaching a mash temperature of 50° C., enzymes were added according to Table 2, and the mashing program (Table 1) was started.

After mashing, the weight of hot mash was adjusted for evaporation losses and immediately transferred to a jacket-heated (78° C.) filter unit type KHS EW14/2CW, equipped with a standard polypropylene mash-filter-cloth and a connection to apply gas pressure. The mash was allowed to settle for 4 minutes. The filter was then pressurized (40 kPa over-pressure) and after a further minute, filtration was started by opening the bottom valve of the filter. The weight of filtrate was automatically monitored every 2 seconds until a total mass of 1050 g was reached. In order to evaluate filtration performance, a modified method described by Vandenbusche et al. (Brewing and Beverage Industry International (3), 2004, p. 14-18) was applied. In brief, linearization of the monitored data was conducted by plotting time/mass over mass. The slope of the linearized data between 200-1000 g was used for calculation of the filtration coefficient (Fk-value). The Fk-value is defined as Fk=η*c/p (η=dyn. Viscosity; a=specific resistance of the filter-cake; c=concentration of insoluble particles; p=pressure). A low Fk-value indicates a good filtration.

In addition to the Fk-value, a second, simpler parameter for filtration performance evaluation, the time until 1000 g of filtrate was collected, was used for illustration.

The filtrate (wort) was analyzed for viscosity, extract density, content of β-glucan>10000 kDa and arabinoxylan.

TABLE 1
Mashing regime applied in the experiment

| Temperature | |
|---|---|
| [° C.] | Duration [min] |
| 50 | 20 |
| 50-64 | 14 |
| 64 | 40 |
| 64-72 | 8 |
| 72 | 20 |
| 72-80 | 8 |
| 80 | 15 |

TABLE 2
Comparison of filtration performance of the tested enzymes and analytical data of the
filtrates obtained. In all trials, Termamyl SC and Neutrase 0.8 L was used at dosage levels of 36 KNU-S/kg
grist and 0.16 AU-N/kg grist, respectively in order to compensate for the reduced
endogenous enzyme activities provided by malt (→ use of 40% un-malted barley). FXU = fungal
xylanase activity units, EGU = endoglucanase activity units, KNU-S = alpha amylase activity units,
AU-N = protease activity units

| | | | Time to | | | |
|---|---|---|---|---|---|---|
| | | | filter 1000 g | Viscosity of | β-glucan content | Arabinoxylan |
| | Dosage | Fk-value | sweet wort | sweet wort | in sweet wort | in sweet wort |
| Enzyme | [per kg grist] | [10\hat{ }6 * s/m\hat{ }2] | [s] | [mPa * s] | [mg/L] | [mg/L] |
| Ultraflo Max | 50 FXU + 140 EGU | 0.279 ± 0.004 | 880 ± 23 | 3.010 ± 0.009 | 150 ± 5 | 529 ± 28 |
| Ultraflo Max | 100 FXU + 280 | 0.248 ± 0.008 | 769 ± 47 | 2.906 ± 0.006 | 61 ± 2 | 519 ± 21 |
| | EGU | | | | | |
| Endo-1,4-beta-xylanase | 100 FXU | | | | | |
| fam. GH10 from | | | | | | |
| Aspergillus aculeatus | | | | | | |
| Endoglucanase fam. | 300 EGU | 0.186 ± 0.001 | 556 ± 3 | 2.674 ± 0.001 | 115 ± 9 | 243 ± 29 |
| GH5_5A-glucanase from | | | | | | |
| Thermoascus | | | | | | |
| aurantiacus | | | | | | |
| Arabinofuranosidase | 7 mg EP | | | | | |
| fam. GH43 from | | | | | | |
| Humicola insolens | | | | | | |

### Results

Mashes were produced from 40% barley and 60% malt and with water to grist ratio of 2.5:1. The premium enzyme blend Ultraflo Max™, used in industry to improve mash separation, which comprises endo-xylanase and a mixture of cellobiohydrolases, endo- and exo-beta-glucanases activity, was added at two dosage levels (50 FXU=fungal xylanase activity units+140 EGU=Endo-glucanase activity units, and 100 FXU+280 EGU based on mass grist) in the beginning of mashing. Mash-filtration performance of these were compared with mashes produced in the same way but with the difference, that the enzyme blend added, comprised endo-1,4-beta-xylanase fam. GH10 from Aspergillus aculeatus, endoglucanase fam. GH5_5 from Thermoascus aurantiacus, and a third enzyme, arabinofuranosidase fam. GH43 from Humicola insolens (SEQ ID:1).

Table 2 demonstrates that the use of the latter combination in mashing, reduced filtration time of sweet wort to ˜63% (when compared to low dosage Ultraflo Max) and ˜72% (high dosage Ultraflo Max), respectively. The better filtration is reflected in the lower viscosity of the filtrate, which is reduced to ˜89% (low dosage Ultraflo Max) and 92% (high dosage of Ultraflo Max), respectively.

The reduction in viscosity can be assigned to the better degradation of high molecular arabinoxylan, which is reduced by ˜53% when the combination of arabinofuranosidase fam. GH43 from Humicola insolens (SEQ ID:1) with endo-1,4-beta-xylanase fam. GH10 from Aspergillus aculeatus is used instead of using the the endo-1,4-beta-xylanase alone.

### Example 4

### Impact of Arabinofuranosidase Fam. GH43 from H. insolens Application in Mashing on Beer Filtration

### Material and Methods

Un-malted barley (64 g) and Pilsner malt (96 g) were milled using a disc-mill at gap distance of 0.2 mm. Calcium-chloride was added to de-salted water to achieve a final concentration of 60 ppm Ca²⁺-ions. After heating the water to 51° C., the grist (room temperature) was added. When reaching a mash temperature of 50° C., enzymes were added according to Table 4 and the mashing program (Table 3) started. Water to grist ratio in mashing was 2.5:1. After mashing, the weight of hot mash was adjusted to 600 g for evaporation losses and immediately filtered through Macherey-Nagel 514¼ filter paper.

Sweet wort was analyzed for density and diluted to 14° Plato using autoclaved, desalted water containing 60 ppm Ca²⁺-ions. Diluted wort was heated to boiling temperature and 0.145 g hops (Hallertauer Tradition, 2012, 17% alpha acids) per 450 g wort added. Wort was boiled for 40 minutes and after boiling, evaporations losses were compensated by addition of autoclaved water. Yeast strain W34/70, propagated overnight was added at a concentration of 2*10⁷ cells/mL wort. Fermentation was conducted at 12° C. for 7 days followed by storage of 5 days on ice. The beer was then de-gassed and centrifuged at 2° C. at 2000 rpm and the supernatant pre-filtered through Whatman 597^{1/2} filter paper in order to remove remaining yeast cells. 50 mL of the pre-filtered beer was then filled into a XK26 column (GE-Healthcare) tempered to 0° C. and equipped with a 0.45 μm cellulose acetate filter. After reaching the temperature equilibrium, 50 kPa pressure (N₂) were applied on the column. The mass of beer filtered over time was monitored automatically every 1 second. The type of filtration applied can be described as a surface filtration, which means that over filtration time, the flow rate is decreasing due to the increasing filter resistance. The first step to evaluate the impact of the individual enzymes, added in mashing, on beer filtration, performance was the calculation of the initial flow rate based on the monitored data. The parameter used to indicate beer filtration performance was obtained by extracting the mass of filtrate and the filtration time from the monitored data reached at the point where the flow rate was half of the initial flow rate. Beer, before and after filtration, was analyzed for content of β-glucan, arabinoxylan and viscosity (at 0° C.).

TABLE 3
Mashing regime applied in the experiment

| Temperature | |
|---|---|
| [° C.] | Duration [min] |
| 50 | 20 |
| 50-64 | 14 |
| 64 | 40 |
| 64-72 | 8 |
| 72 | 20 |
| 72-80 | 8 |
| 80 | 15 |

TABLE 4
Comparison of beer filtration performance of the tested enzymes and analytical data of the
un- and filtrates obtained. In all trials, Termamyl SC and Neutrase 0.8 L was used at dosage levels
of 36 KNU-S/kg grist and 0.16 AU-N/kg grist, respectively in order to compensate for the reduced
endogenous enzyme activities provided by malt (→ use of 40% un-malted barley). FXU = fungal
xylanase activity units, EGU = endoglucanase activity units, KNU-S = alpha amylase activity units,
AU-N = protease activity units.

| | | Mass of | | | | | | | |
|---|---|---|---|---|---|---|---|---|---|
| | | filtrate | Length | | | | | | |
| | | collected | of period | | | | | | |
| | | after the | until | | | | | | |
| | | period until | initial | | Viscosity | β- | | Arabino- | Arabino- |
| | | initial flow | flow rate | | at | glucan | | xylan | xylan |
| | | rate | decreased | Viscosity at | 0 C.° of | content | β-glucan | content in | glucan |
| | | decreased | to | 0 C.° of beer | beer | in beer | content in | beer | content in |
| | | to ½* initial | ½* initial | before | after | before | beer after | before | beer after |
| | Dosage | flow rate | flow rate | filtration | filtration | filtration | filtration | filtration | filtration |
| Enzyme | (per kg grist) | (g) | (s) | [mPa * s] | [mPa * s] | [mg/L] | [mg/L] | [mg/L] | [mg/L] |
| Ultraflow Max | 100 | 18.35 ± 0.42 | 58 ± 2 | 3.156 ± 0.007 | 3.070 ± 0.004 | 47 ± 2 | 32 ± 3 | 375 ± 15 | 308 ± 21 |
| | FXU + | | | | | | | | |
| | 280 | | | | | | | | |
| | EGU | | | | | | | | |
| Endo-1,4-beta- | 100 | 21.49 ± 0.66 | 66 ± 1 | 3.016 ± 0.009 | 2.996 ± 0.006 | 87 ± 3 | 56 ± 1 | 187 ± 8 | 155 ± 13 |
| xylanase fam. GH10 | FXU | | | | | | | | |
| from Aspergillus | | | | | | | | | |
| aculeatus | | | | | | | | | |
| Endoglucanase fam. | 300 | | | | | | | | |
| GH5_5A-glucanase | EGU | | | | | | | | |
| from Thermoascus | | | | | | | | | |
| aurantiacus | | | | | | | | | |
| Aranbinofuranosidase | 7 mg | | | | | | | | |
| fam. GH43 from | EP | | | | | | | | |
| Humicola insolens | | | | | | | | | |

### Results

Beers were produced from 40% barley and 60% malt with an original gravity of 14° Plato. The premium enzyme blend Ultraflo Max, used in industry to improve mash- and beer separation, which comprises endo-xylanase- and a mixture of cellobiohydrolases-, endo- and exo-beta-glucanases activity, was dosed at the beginning of mashing at the upper dosage limit normally applied in industry (100 FXU=fungal xylanase activity units+280 EGU=Endo-glucanase activity units based on mass grist) to serve as challenging benchmark. Filtration performance was compared with beers produced in the same way but with the difference, that the enzyme blend added, comprised endo-1,4-beta-xylanase fam. GH10 from Aspergillus aculeatus, endoglucanase fam. GH5_5 from Thermoascus aurantiacus, and a third enzyme, arabinofuranosidase fam. GH43 from Humicola insolens (AraFGH43). Table 2 demonstrates, that the use of the latter combination in mashing, improved beer filtration by ˜17% (based on mass of filtrate collected after the period until initial flow rate decreased to ½*initial flow rate) or ˜14% (based on length of period until initial flow rate decreased to ½*initial flow rate). The better filtration has its origin in the lower viscosity enabling a faster initial flow and i.e. in the lower content of total hemicellulose (sum of β-glucan+arabinoxylan), which reduced the saturation (clocking) of the membrane filter with filtrate residue per mass unit filtrate (saturation of the membrane in the range of ˜63-82 mg hemicellulose).

1. A method of reducing the viscosity in a brewing process comprising the steps of:
(a) preparing a mash from malt and adjunct, and
(b) adding an arabinofuranosidase GH43 to the mash.

2. The method according to claim 1, wherein the adjunct is selected from the group consisting of barley and wheat.

3. The method according to claim 1, wherein additionally a beta-glucanase is added to the mash.

4. The method according to claim 1, wherein additionally a xylanase is added to the mash.

5. The method according to claim 1, wherein the arabinofurosidase has at least 70% identity to the sequence shown in SEQ ID NO: 1.

6. The method according to claim 1, wherein the mash has a water/grist ratio of 2.0:1.0 to 3.0:1.0 (w/w).

7. The method according to claim 1, wherein the adjunct is at least 10% (w/w) of the total weight of adjunct and malt.

8. The method according to claim 1, wherein the mashing comprises an incubation step at a temperature in the range of from 45° C. to 60° C.

9. The method according to claim 1, wherein additionally a protease is added to the mash.

10. The method according to claim 1, wherein additionally a pullulanase is added to the mash.

11. The method according to claim 1, wherein additionally a lipase is added to the mash.

12. The method according to claim 1, wherein additionally an alpha-amylase is added to the mash.

13. The method according to claim 1, wherein the mash is filtered to obtain a wort.

14. The method according to claim 13, wherein the wort is fermented to obtain a beer.

15. The method according to claim 1, wherein the arabinofuranosidase GH43 is added in an amount of from 0.5 to 10.0 mg enzyme protein per kg grist.

16. The method according to claim 1, wherein the arabinofuranosidase GH43 has activity towards di-substituted xyloses.


SEQUENCE LISTING (2 sequences)

SEQ ID NO: 1 - protein, 531 aa, Humicola insolens
QASTFTNPVLWEDHPDLEVFRVGSVFYYSSSTFAYSPGAPVLKSYDLVHWTPVTHSVPRLNFGSNYDLPSGTPGAYVKGIWASTLRYRRSNDRFYWYGCVEGRTYLWTSPGGNALANNGEVPPSAWNWQHTATIDNCYYDAGLLIDDDDTMYIAYGNPTINVAQLSPDGTRQVRVQQRVYAHPQGQTVEGARMYKIRGNYYILVTRPADAEYVLRSTTGSPFGPYEARTLVSRIQGPLANAGFAHQGGIVDAPDGTWHYVAFMDAYPGGRIPVVAPLRWTADGWPEVVTDSQGRWGTSYPIPVRGAKNATEGLASTDLDEFRGTRFSEHWEWNHNPDTSKFTLLGGNEGGLILRTATVTGDLFAARNTLTRRIAGPKASGIFRLDVRGMRDGDRAGAVLFRDRAAYIGVWKQGNEARIVMVDDLRLNEDGWRTASTGRVAANGPVIDTNAQQDIWLRIDADITPAFGTNTERTTTFYYSIDGGRTYTRLGPAFAMTNSWRYFTGYRFGVFNFSTKSLGGEVKVKGFKMNMI

SEQ ID NO: 2 - protein, 627 aa, Meripilus giganteus
VTVTVNKNPSHTVPSTLYGLMFEDINHSGDGGLYAELLQNRAFQQVTPNTAAALAAWHPISNAKLAVIQDPSPVSNALPNSLQFSVPSGSSGRVGFTNEGFWGIKVDSTWTYKASLFFRFPTSSSFSGALTVGLQTNAGRVLAQNSTQIRGTTTKWTQINLELHPTASAPDVSNSFFVTIDGAAGAGQTINFAMFSLFPPTFKNRPNGLRADIAETLAEMGPSFFRFPGGNNLEGQTTATRWQWNATVGSLLDRPGRVGDWGYVNTDGLGLLEYLQFFEDTGMEPIMAVWAGYSLGGTSLAENQLAPYIQQAIDQINFVIGDPAKSAPAALRASLGHPEPFTLRFVEVGNEDFFAAGSYPYRWHDFVTALQAQFPQIRFIATTNAWNPVLSPVPQSYDVHVYQTPTWFYQNAFYYDGFQRNGTTYFEGEYAAISTNANDLFGTVADGRLAFPTVQSATGEAAFMTGLERNSDIVFAASYAPLLQHVNSTQWTPDLVSYDAGSVIKSTSFFAQKLFALNKGDQYLPSTLPTNGGTLHWSITRASSSGKTFIKIANAGSSAQSLTFQLTQFNSVSSTGTLQVLTGPETASNTPEAPQAIVPKTSTIGTGKTFTYNAPAFSVSVITVTTN